(19) United States
(12) Patent Application Publication
(10) Pub. No.: US 2023/0151067 A1
(43) Pub. Date: May 18, 2023

(54) Preparation Method of Surimi-Low-Molecular-Weight Antifreeze Peptide

(71) Applicant: Dalian Minzu University, Dalian City (CN)

(72) Inventors: Tingting LI, Dalian City (CN); Zhe XU, Dalian City (CN); Jianrong Li, Dalian City (CN); Zhixuan Zhu, Dalian City (CN); Lingyu Han, Dalian City (CN); Yu Zou, Dalian City (CN); Kun Ma, Dalian City (CN)

(21) Appl. No.: 17/740,837

(22) Filed: May 10, 2022

(30) Foreign Application Priority Data
Nov. 16, 2021 (CN) 202111354737.X

(51) Int. Cl. C07K 14/46; C12P 21/06

(52) U.S. Cl. CPC C07K 14/461; C12P 21/06

(57) ABSTRACT

The present disclosure provides a preparation method of a surimi low-molecular-weight antifreeze peptide, and relates to the technical field of processing of aquatic products. The preparation method includes the following steps: step 1, with Larimichthys crocea as a raw material, decapitating and gutting the L. crocea, crushing the L. crocea into a surimi, decalcifying the surimi, extracting water-soluble crude protein with water, and freeze-drying to obtain the water-extracted crude protein powder of L. crocea; step 2, redissolving the water-extracted crude protein powder of L. crocea obtained in step 1 with water, and enzymatically hydrolyzing it with protease to obtain an enzymatic hydrolysate; and step 3, sonicating and dialyzing the enzymatic hydrolysate obtained in step 2 to obtain a dialysate with a molecular weight no more than 3,500 D, and freeze-drying the dialysate to obtain the surimi low-molecular-weight antifreeze peptide.

### CROSS REFERENCE TO RELATED APPLICATION(S)

This patent application claims the benefit and priority of Chinese Patent Application No. 202111354737.X, entitled “PREPARATION METHOD OF SURIMI LOW-MOLECULAR-WEIGHT ANTIFREEZE PEPTIDE” filed on Nov. 16, 2021, the disclosure of which is incorporated by reference herein in its entirety as part of the present application.

### TECHNICAL FIELD

The present disclosure relates to the technical field of processing of aquatic products, and in particular to a preparation method of a surimi low-molecular-weight antifreeze peptide.

### BACKGROUND ART

At present, cold chain transportation is often used to increase sales of fish or surimi products. However, taste of fish flesh may decrease, and proteins and fats in fish flesh may be oxidized during the frozen storage of the fish or surimi products, leading to decreased value and quality of the fish or surimi products. In order to maintain quality of the fish or surimi products, antifreeze agents such as sucrose and maltodextrin will be added usually, which will change the taste and quality of the fish or surimi products. Therefore, looking for a novel, efficient, healthy, and natural antifreeze agent without changing quality of fish flesh becomes a major problem during the storage and transportation of the fish or surimi products.

Larimichthys crocea is a nutritious aquatic product, most of which live in mid-to-lower waters. L. crocea protein has high nutritional value and excellent antioxidant, anti-inflammatory and antitumor activity. Remarkably, L. crocea has certain medicinal value, which is a raw material for some Chinese patent medicine and western medicine. “Fish gelatin bead” prepared by frying and roasting fish swimbladder is indicated for treatment of peptic ulcer, renal calculi, and the like. There is increasing evidence that aquatic products with high protein content are digested, enzymatically hydrolyzed, and converted into small bioactive peptides that are easy to be absorbed by human body, and thus eating aquatic products with high protein content benefits the human body.

At present, in studies of antifreeze activity, some antifreeze proteins can change ice crystal growth by inhibiting thermal hysteresis and ice recrystallization, thereby reducing damage of organelles in fish or surimi and exhibiting antifreeze activity. However, because antifreeze proteins are not easily absorbed, their structures with effective antifreeze activity are not easily exposed, water solubility of them are poor, antifreeze proteins are not easy to be used in fish or surimi products and difficult to achieve an objective of application in industrial production. Some studies have indicated that high molecular weight peptides have poorer activity than low molecular weight peptides. Most existing extraction techniques of antifreeze peptides use fish skin, fish flesh, or fish bone as a raw material, but rarely extracted from a whole fish. However removement of fish skin and fish bone during extraction are too complicated in operation to be suitable for actual production.

### SUMMARY

An objective of the present disclosure is to provide a preparation method of a surimi low-molecular-weight antifreeze peptide in order to solve the above problems present in prior art. L. crocea low-molecular-weight peptide products with antifreeze activity can be obtained by the preparation method provided by the present disclosure.

To achieve the above objective, the present disclosure provides a following solution:

The present disclosure provides a preparation method of a surimi low-molecular-weight antifreeze peptide, including the following steps:

step 1, preparation of water-extracted crude protein powder of L. crocea

with L. crocea as a raw material, decapitating and gutting the L. crocea, crushing the L. crocea into a surimi, decalcifying the surimi, extracting water-soluble crude protein with water, and freeze-drying to obtain the water-extracted crude protein powder of L. crocea;

step 2, enzymatic hydrolysis

redissolving the water-extracted crude protein powder of L. crocea obtained in step 1 with water, and enzymatically hydrolyzing the water-extracted crude protein powder of L. crocea with protease to obtain an enzymatic hydrolysate;

step 3, sonication and small molecules retention

sonicating and dialyzing the enzymatic hydrolysate obtained in step 2 to obtain a dialysate with a molecular weight no more than 3,500 D, and freeze-drying the dialysate to obtain the surimi low-molecular-weight antifreeze peptide.

Further, in step 1, the decalcifying is conducted in an EDTA decalcifying solution.

Further, the EDTA decalcifying solution has a concentration of 0.25 mol/L.

Further, in step 1, decalcified surimi and water have a mass ratio of 1:(5-10) when the water-soluble crude protein is extracted with water.

Further, in step 2, the water-extracted crude protein powder of L. crocea and the water have a mass ratio of 1:(40-120) when redissolving with water.

Further, in step 2, the protease is pepsin or trypsin.

Further, in step 2, the enzymatic hydrolysis is conducted at 30 to 50° C. for 1 to 6 h.

Further, in step 3, the sonication is conducted at 100 to 200 W for 5 to 30 min, 200 to 400 W for 5 to 30 min, and 100 to 200 W for 5 to 30 min.

Further, in step 3, the dialyzing is constructed by using a 500 to 3,500 D dialysis bag.

Further, in step 1, the freeze-drying is conducted by gradient temperature change method: stage 1: pre-freezing at −65 to −50° C. for 3 to 5 h; stage 2: freezing at −50 to −40° C. for 1 to 3 h; stage 3: freezing at −40 to −25° C. for 1 to 2 h; stage 4: freezing at −25 to −5° C. for 1 to 2 h; stage 5: drying at 5 to 15° C. for 1 to 2 h; stage 6: drying at 15 to 20° C. for 1 to 2 h; and stage 7: drying at 20 to 25° C. for 1 to 2 h.

The present disclosure provides the following technical effects:

(1) L. crocea is a common aquatic product derived from a wealth of sources. The L. crocea, as a raw material, is easily available, safe and free of side effect, significantly reduces production costs, and meets the requirement for mass production.

(2) In the present disclosure, a combination of biological enzymatic hydrolysis with variable-frequency sonication technology achieves an objective of effective extraction of low molecular weight peptides, featuring easy operation and mild production conditions.

(3) In the present disclosure, freeze-drying of the water-soluble crude protein extracted can improve the antifreeze activity of the surimi low-molecular-weight antifreeze peptide.

(4) In the present disclosure, extraction of antifreeze peptides from bones and fish flesh of the L. crocea together simplifies the processing of the fish, while the surimi low-molecular-weight antifreeze peptide prepared has a better antifreeze activity.

(5) In the present disclosure, addition of only trace amounts of the surimi low-molecular-weight antifreeze peptide can achieve and even outweigh the effect of conventional antifreezes obtain by addition of a large amount of sucrose; moreover, the peptide, which is derived from aquatic products, inhibits fishy smell, increases scent, and has a better effect than conventional antifreezes.

### BRIEF DESCRIPTION OF THE DRAWINGS

To describe the technical solutions in the examples of the present disclosure or in the prior art more clearly, the drawings required for describing the examples will be briefly described. Apparently, the drawings in the following description show merely some examples of the present disclosure, and a person of ordinary skill in the art may still derive other drawings from these drawings without creative efforts.

FIG. 1 illustrates detection results of drip loss of fish flesh after different treatments;

FIG. 2 illustrates detection results of odor changes of fish flesh after different treatments.

### DETAILED DESCRIPTION OF THE EMBODIMENTS

Various exemplary embodiments of the present disclosure will now be described in detail. The detailed description should not be considered as a limitation of the present disclosure, but should be understood as a more detailed description of certain aspects, features, and embodiments of the present disclosure.

It should be appreciated that terms described in the present disclosure are only intended to describe specific embodiments and are not intended to limit the present disclosure. In addition, for numerical range in the present disclosure, it should be appreciated that each intermediate value between the upper limit and the lower limit of the range is also specifically disclosed. Each smaller range between any stated value or intermediate value in the stated range and each smaller range between any other stated value or intermediate value in the stated range is also included in the present disclosure. The upper and lower limits of these smaller ranges can independently be included or excluded from the range.

Unless otherwise stated, all technical and scientific terms used herein have the same meaning as commonly understood by those of ordinary skill in the art described in the present disclosure. Although the present disclosure describes only preferred methods and materials, any methods and materials similar or equivalent to those described herein can also be used in practice or testings of the present disclosure. All literature mentioned in this specification are incorporated by reference to disclose and describe methods and/or materials related to the documents. In case of conflict with any incorporated literature, the content of this specification shall prevail.

It is obvious to those skilled in the art that various modifications and variations can be made to specific implementations of the present specification without departing from the scope or spirit of the present disclosure. Other embodiments derived from the specification of the present disclosure will be obvious to the skilled person in the art. The specification and examples of the present disclosure are merely exemplary.

As used herein, “including”, “having”, “comprising”, etc. are all open-ended terms, which means including but not limited to.

### Example 1

(1) Preparation of Water-Extracted Crude Protein of L. crocea

With L. crocea as a raw material, the L. crocea was decapitated, gutted, and crushed into a surimi; the surimi was decalcified in 0.25 mol/L of EDTA decalcifying solution for 12 h, and washed with water until neutral, to obtain a decalcified surimi. 550 g of the decalcified surimi was homogenized with 5,500 g of water in a homogenizer at 10,000 rpm for 10 min and stirred at 4° C. and 40 rpm for 6 h to extract proteins; the proteins were centrifuged at 10,000 G for 30 min to obtain a water-extracted crude protein solution.

(2) Freeze-Drying

The water-extracted crude protein solution obtained in step 1 was freeze-dried by gradient temperature change method, and the time-temperature program of the freeze-drying was set as follows: stage 1: pre-freezing at −60° C. for 3 h; stage 2: freezing at −50° C. for 3 h; stage 3: freezing at −40° C. for 2 h; stage 4: freezing at −5° C. for 1 h; stage 5: drying at 10° C. for 2 h; stage 6: drying at 20° C. for 2 h; and stage 7: drying at 25° C. for 2 h. Vacuuming was conducted at stage 2, and a constant temperature was held at stage 7. The starting temperature of the vacuum pump was −65° C., the separator temperature was set at −23° C., and 10 g of water-extracted crude protein powder of L. crocea was obtained.

(3) Preparation of L. crocea Antifreeze Peptide

The water-extracted crude protein powder of L. crocea obtained in step 2 was redissolved with 800 g of water, and enzymatically hydrolyzed with 5,000 U/g trypsin (relative to the water-extracted crude protein powder) at 37° C. for 5 h to obtain an enzymatic hydrolysate.

(4) Variable-Frequency Sonication

The enzymatic hydrolysate obtained in step 3 was subjected to variable-frequency sonication: at 100 W for 5 min, 200 W for 5 min, and 100 W for 5 min; and a mixed peptide solution of L. crocea dissolved homogeneously in water was obtained.

(5) Retention of Small Molecules

The mixed peptide solution of L. crocea obtained in step 4 was placed in a 3,000 D dialysis bag, dialyzed with double distilled water for a total of 24 h (the consumption of the double distilled water was 100-fold that of the mixed peptide solution of L. crocea, and the double distilled water was changed every 12 h) to obtain a dialysate.

(6) Freeze-Drying

The dialysate obtained in step 5 was freeze-dried as follows: stage 1: pre-freezing at −60° C. for 3 h; stage 2: freezing at −50° C. for 3 h; stage 3: freezing at −40° C. for 2 h; stage 4: freezing at −5° C. for 1 h; stage 5: drying at 10° C. for 2 h; stage 6: drying at 20° C. for 2 h; and stage 7: drying at 25° C. for 2 h. Vacuuming was conducted at stage 2, and a constant temperature was held at stage 7. The starting temperature of the vacuum pump was −65° C., the separator temperature was set at −23° C., and 6 g of surimi low-molecular-weight antifreeze peptide was obtained.

### Example 2

(1) Preparation of Water-Extracted Crude Protein of L. crocea

With L. crocea as a raw material, the L. crocea was decapitated, gutted, and crushed into a surimi; the surimi was decalcified in 0.25 mol/L of EDTA decalcifying solution for 12 h, and washed with water until neutral, to obtain a decalcified surimi. 450 g of the decalcified surimi was homogenized with 2,250 g of water in a homogenizer at 10,000 rpm for 10 min and stirred at 4° C. and 40 rpm for 6 h to extract proteins; the proteins were centrifuged at 10,000 G for 30 min to obtain a water-extracted crude protein solution.

(2) Freeze-Drying

The water-extracted crude protein solution obtained in step 1 was freeze-dried by gradient temperature change method, and the time-temperature program of the freeze-drying was set as follows: stage 1: pre-freezing at −65° C. for 3 h; stage 2: freezing at −50° C. for 1 h; stage 3: freezing at −40° C. for 1 h; stage 4: freezing at −25° C. for 1 h; stage 5: drying at 5° C. for 1 h; stage 6: drying at 15° C. for 1 h; and stage 7: drying at 20° C. for 1 h. Vacuuming was conducted at stage 2, and a constant temperature was held at stage 7. The starting temperature of the vacuum pump was −65° C., the separator temperature was set at −23° C., and 8 g of water-extracted crude protein powder of L. crocea was obtained.

(3) Preparation of L. crocea Antifreeze Peptide

The water-extracted crude protein powder of L. crocea obtained in step 2 was redissolved with 320 g of water, and enzymatically hydrolyzed with 5,000 U/g pepsin (relative to the content of protein in the water-extracted crude protein powder) at 30° C. for 1 h to obtain an enzymatic hydrolysate.

(4) Variable-Frequency Sonication

The enzymatic hydrolysate obtained in step 3 was subjected to variable-frequency sonication: at 100 W for 5 min, 200 W for 5 min, and 100 W for 5 min; and a mixed peptide solution of L. crocea dissolved homogeneously in water was obtained.

(5) Retention of Small Molecules

The mixed peptide solution of L. crocea obtained in step 4 was placed in a 500 D dialysis bag, dialyzed with double distilled water for a total of 24 h (the consumption of the double distilled water was 100-fold that of the mixed peptide solution of L. crocea, and the double distilled water was changed every 12 h) to obtain a dialysate.

(6) Freeze-Drying

The dialysate obtained in step 5 was freeze-dried as follows: stage 1: pre-freezing at −65° C. for 3 h; stage 2: freezing at −50° C. for 1 h; stage 3: freezing at −40° C. for 1 h; stage 4: freezing at −25° C. for 1 h; stage 5: drying at 5° C. for 1 h; stage 6: drying at 15° C. for 1 h; and stage 7: drying at 20° C. for 1 h. Vacuuming was conducted at stage 2, and a constant temperature was held at stage 7. The starting temperature of the vacuum pump was −65° C., the separator temperature was set at −23° C., and 4.5 g of surimi low-molecular-weight antifreeze peptide was obtained.

(1) Preparation of Water-Extracted Crude Protein of L. crocea

With L. crocea as a raw material, the L. crocea was decapitated, gutted, and crushed into a surimi; the surimi was decalcified in 0.25 mol/L of EDTA decalcifying solution for 12 h, and washed with water until neutral, to obtain a decalcified surimi. 800 g of the decalcified surimi was homogenized with 8,000 g of water in a homogenizer at 10,000 rpm for 10 min and stirred at 4° C. and 40 rpm for 6 h to extract proteins; the proteins were centrifuged at 10,000 G for 30 min to obtain a water-extracted crude protein solution.

(2) Freeze-Drying

The water-extracted crude protein solution obtained in step 1 was freeze-dried by gradient temperature change method, and the time-temperature program of the freeze-drying was set as follows: stage 1: pre-freezing at −50° C. for 5 h; stage 2: freezing at −40° C. for 3 h; stage 3: freezing at −25° C. for 2 h; stage 4: freezing at −5° C. for 2 h; stage 5: drying at 15° C. for 2 h; stage 6: drying at 20° C. for 2 h; and stage 7: drying at 25° C. for 2 h. Vacuuming was conducted at stage 2, and a constant temperature was held at stage 7. The starting temperature of the vacuum pump was −65° C., the separator temperature was set at −23° C., and 14.5 g of water-extracted crude protein powder of L. crocea was obtained.

(3) Preparation of L. crocea Antifreeze Peptide

The water-extracted crude protein powder of L. crocea obtained in step 2 was redissolved with 1,740 g of water, and enzymatically hydrolyzed with 5,000 U/g trypsin (relative to the content of protein in the water-extracted crude protein powder) at 50° C. for 6 h to obtain an enzymatic hydrolysate.

(4) Variable-Frequency Sonication

The enzymatic hydrolysate obtained in step 3 was subjected to variable-frequency ultrasound: at 200 W for 30 min, 400 W for 30 min, and 200 W for 30 min; and a mixed peptide solution of L. crocea dissolved homogeneously in water was obtained.

(5) Retention of Small Molecules

The mixed peptide solution of L. crocea obtained in step 4 was placed in a 3,500 D dialysis bag, dialyzed with double distilled water for a total of 24 h (the consumption of the double distilled water was 100-fold that of the mixed peptide solution of L. crocea, and the double distilled water was changed every 12 h) to obtain a dialysate.

(6) Freeze-Drying

The dialysate obtained in step 5 was freeze-dried as follows: stage 1: pre-freezing at −50° C. for 5 h; stage 2: freezing at −40° C. for 3 h; stage 3: freezing at −25° C. for 2 h; stage 4: freezing at −5° C. for 2 h; stage 5: drying at 15° C. for 2 h; stage 6: drying at 20° C. for 2 h; and stage 7: drying at 25° C. for 2 h. Vacuuming was conducted at stage 2, and a constant temperature was held at stage 7. The starting temperature of the vacuum pump was −65° C., the separator temperature was set at −23° C., and 9 g of surimi low-molecular-weight antifreeze peptide was obtained.

### Comparative Example 1

This Comparative Example was the same as Example 1, and the difference was that in the comparative example, L. crocea was bone removed, decapitated, and gutted together in step 1, and only fish flesh was left. In this comparative example, 5.6 g of surimi low-molecular-weight antifreeze peptide was prepared.

### Comparative Example 2

This Comparative Example was the same as Example 1, and the difference was that in the comparative example, L. crocea was flesh removed, decapitated, and gutted together in step 1, and only fish bones were left. In this comparative example, 0.4 g of surimi low-molecular-weight antifreeze peptide was prepared.

### Comparative Example 3

This Comparative Example was the same as Example 1, and the difference was that in the comparative example, the water-extracted crude protein solution obtained in step 1 was not freeze-dried, but enzymatically hydrolyzed with an enzyme directly. In this comparative example, 6 g of surimi low-molecular-weight antifreeze peptide was prepared.

### Comparative Example 4

This Comparative Example was the same as Example 1, and the difference was that in the comparative example, there was a lack of step 4. In this comparative example, 5.2 g of surimi low-molecular-weight antifreeze peptide was prepared.

### Comparative Example 5

This Comparative Example was the same as Example 1, and the difference was that in the comparative example, the sonication in step 4 was conducted at 100 W for 15 min. In this comparative example, 5.6 g of surimi low-molecular-weight antifreeze peptide was prepared.

Detection of Antifreeze Activity

Fresh fish flesh derived from the same part was cut into evenly sized fish fillets (1*1*0.5 cm), and the fish fillets were treated according to the treatment methods in Table 1, respectively. Then, texture properties in the texture profile analysis (TPA) were detected by a texture analyzer: a p/50 probe was used; the speed was 1 mm/s before, during, and after detection; the displacement was 2 mm; the trigger force was 5×g. Their hardness, springiness, cohesiveness, chewiness, and resilience were obtained. The detection results are shown in Table 2. From Table 2, the surimi low-molecular-weight antifreeze peptide extracted from fish bones or flesh alone has poor antifreeze activity, but that extracted together from fish bones and flesh can achieve excellent antifreeze activity; freeze-drying of the water-soluble crude protein extracted can improve the antifreeze activity of the surimi low-molecular-weight antifreeze peptide in terms of springiness, chewiness, and resilience.

TABLE 1

| Experimental | |
|---|---|
| group | Treatment |
| Fresh | No treatment |
| Blank | Freezed at −20° C. for 4 h and thawed at 4° C. for 4 h was |
| | recorded as a freeze-thaw cycle; underwent three freeze- |
| | thaw cycles. |
| Example | Fish fillets was soaked in the surimi low-molecular-weight |
| 1/2/3 | antifreeze peptide solution (2 mg/mL) prepared in Example |
| | 1/2/3 at 4° C. for 4 h, took out, and wiped off the liquid |
| | on its surface; frozen at −20° C. for 4 h and thawed at 4° |
| | C. for 4 h, which was recorded as a freeze-thaw cycle; |
| | underwent three freeze-thaw cycles. |
| Comparative | Fish fillets was soaked in the surimi low-molecular-weight |
| Example | antifreeze peptide solution (2 mg/mL) prepared in Compar- |
| 1/2/3 | ative Example 1/2/3 at 4° C. for 4 h, took out, and wiped |
| | off the liquid on its surface; frozen at −20° C. for 4 h |
| | and thawed at 4° C. for 4 h, which was recorded as a |
| | freeze-thaw cycle; underwent three freeze-thaw cycles. |
| Sucrose | Fish fillets was soaked in a commercial antifreeze solution |
| | (4% (w/w) sucrose solution) at 4° C. for 4 h, took out, |
| | and wiped off the liquid on its surface; frozen at −20° C. |
| | for 4 h and thawed at 4° C. for 4 h, which was recorded |
| | as a freeze-thaw cycle; underwent three freeze-thaw cycles. |

TABLE 2

| | Hard- | Spring- | Cohesive- | Chewi- | Resil- |
|---|---|---|---|---|---|
| | ness | iness | ness | ness | ience |
| Item | (g) | (mm) | (g) | (mJ) | (mm) |
| Fresh | 51.5 | 1.12 | 0.49 | 22.0 | 0.28 |
| Blank | 36.2 | 0.80 | 0.36 | 18.1 | 0.20 |
| Sucrose | 42.1 | 0.92 | 0.42 | 18.0 | 0.17 |
| Example 1 | 48.9 | 1.08 | 0.48 | 21.2 | 0.24 |
| Example 2 | 47.7 | 1.02 | 0.45 | 21.0 | 0.22 |
| Example 3 | 46.0 | 1.00 | 0.43 | 20.8 | 0.22 |
| Comparative | 40.2 | 0.82 | 0.42 | 20.2 | 0.20 |
| Example 1 | | | | | |
| Comparative | 43.8 | 0.96 | 0.40 | 20.1 | 0.24 |
| Example 2 | | | | | |
| Comparative | 48.1 | 0.94 | 0.47 | 18.2 | 0.20 |
| Example 3 | | | | | |

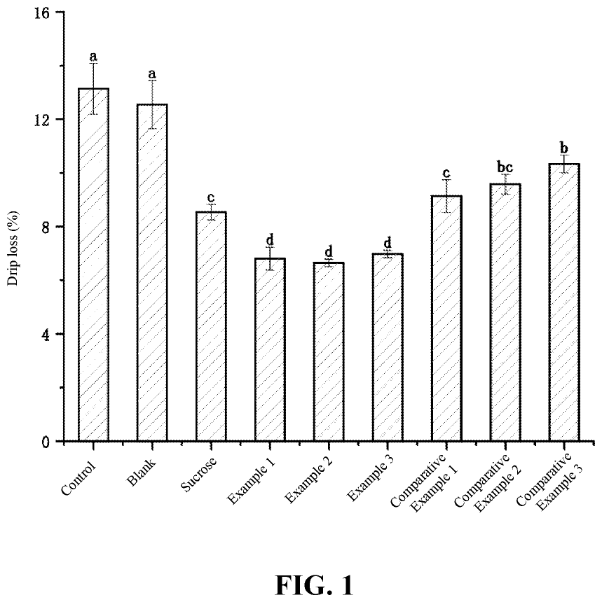

Fresh fish flesh derived from the same part was cut into evenly sized fish fillets, and the fish fillets were weighed (approximately 5 g) and treated according to the treatment methods in Table 1, respectively. Then, the liquid on their surfaces was wiped off and the obtained fish fillets were weighed again to obtain their drip losses. Detection results are shown in FIG. 1. From FIG. 1, the surimi low-molecular-weight antifreeze peptide extracted from fish bones or flesh alone has poor water holding capacity, but that extracted together from fish bones and flesh can achieve excellent water holding capacity; freeze-drying of the water-soluble crude protein extracted can improve the antifreeze activity of the surimi low-molecular-weight antifreeze peptide in terms of water holding capacity.

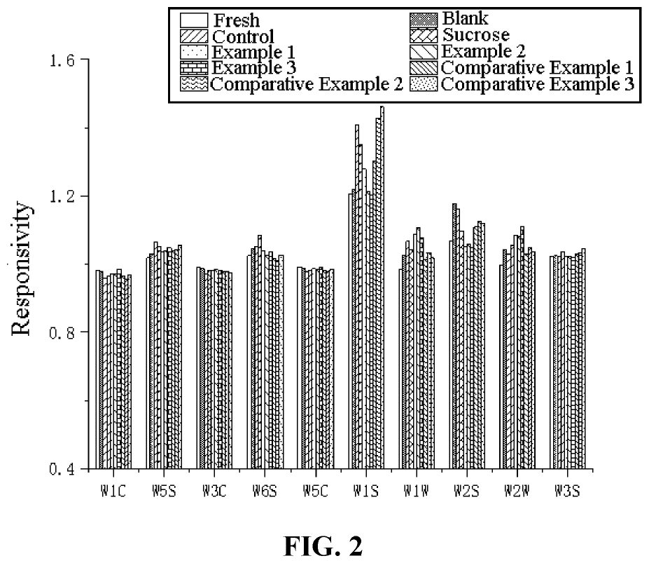

Fresh fish flesh derived from the same part was cut into evenly sized fish fillets, and the fish fillets were weighed (approximately 5 g) and treated according to the treatment methods in Table 1, respectively. Then, analysis was conducted by German PEN3 Portable Electronic Nose sensor. Responsivity of fish fillets to ten types of substances obtained are shown in Table 3. Detection results are shown in FIG. 2. From FIG. 2, the surimi low-molecular-weight antifreeze peptide extracted from fish bones or flesh alone shows poor inhibition of fishy smell, but that extracted together from fish bones and flesh can achieve effects of removing the fishy smell and increasing scent, so as to better inhibit the fishy smell of aquatic products, increase the scent of fish, improve the entire scent, and contribute to harmony of the entire scent; freeze-drying of the water-soluble crude protein extracted can improve the antifreeze activity of the surimi low-molecular-weight antifreeze peptide in terms of maintenance of flavor.

TABLE 3

| Name of the sensor | Performance profile |
|---|---|
| W1C | Aromatic components(Benzenes) |
| W5S | Nitrogen oxides |
| W3C | Ammoniac compounds and aromatic components |
| W6S | Hydrides |
| W5C | Short-chain alkane aromatic components |
| W1S | Methyls |
| W1W | Inorganic sulfides |
| W2S | Alcohols, aldehydes, and ketones |
| W2W | Organic sulfides and aromatic components |
| W3S | Long-chain alkanes |

The above examples are only intended to describe some embodiments of the present disclosure and not intended to limit the scope of the present disclosure. Various alterations and improvements made by those of ordinary skill in the art based on the technical solution of the present disclosure without departing from the design spirit of the present disclosure shall fall within the scope of the appended claims of the present disclosure.

What is claimed is:

1. A preparation method of a surimi low-molecular-weight antifreeze peptide, comprising following steps:
step 1, preparation of water-extracted crude protein powder of Larimichthys crocea
with Larimichthys crocea as a raw material, decapitating and gutting the Larimichthys crocea, crushing the Larimichthys crocea into a surimi, decalcifying the surimi, extracting water-soluble crude protein with water, and freeze-drying to obtain the water-extracted crude protein powder of Larimichthys crocea;
step 2, enzymatic hydrolysis
redissolving the water-extracted crude protein powder of Larimichthys crocea obtained in step 1 with water, and enzymatically hydrolyzing the water-extracted crude protein powder of Larimichthys crocea with protease to obtain an enzymatic hydrolysate;
step 3, sonication and retention of small molecules
sonicating and dialyzing the enzymatic hydrolysate obtained in step 2 to obtain a dialysate with a molecular weight no, more than 3,500 D, and freeze-drying the dialysate to obtain the surimi low-molecular-weight antifreeze peptide.

2. The preparation method according to claim 1, wherein in step 1, the decalcifying is conducted in an EDTA decalcifying solution.

3. The preparation method according to claim 2, wherein the EDTA decalcifying solution has a concentration of 0.25 mol/L.

4. The preparation method according to claim 1, wherein in step 1, the decalcified surimi and the water have a mass ratio of 1:(5-10) when the water-soluble crude protein is extracted with the water.

5. The preparation method according to claim 1, wherein in step 2, the water-extracted crude protein powder of Larimichthys crocea and the water have a mass ratio of 1:(40-120) when redissolving with the water.

6. The preparation method according to claim 1, wherein in step 2, the protease is pepsin or trypsin.

7. The preparation method according to claim 1, wherein in step 2, the enzymatic hydrolysis is conducted at 30 to 50° C. for 1 to 6 h.

8. The preparation method according to claim 1, wherein in step 3, the sonication is conducted at 100 to 200 W for 5 to 30 min, 200 to 400 W for 5 to 30 min, and 100 to 200 W for 5 to 30 min.

9. The preparation method according to claim 1, wherein in step 3, the dialyzing is conducted by using a 500 to 3,500 D dialysis bag.

10. The preparation method according to claim 1, wherein in step 1, the freeze-drying is conducted by gradient temperature change method: stage 1: pre-freezing at −65 to −50° C. for 3 to 5 h; stage 2: freezing at −50 to −40° C. for 1 to 3 h; stage 3: freezing at −40 to −25° C. for 1 to 2 h; stage 4: freezing at −25 to −5° C. for 1 to 2 h; stage 5: drying at 5 to 15° C. for 1 to 2 h; stage 6: drying at 15 to 20° C. for 1 to 2 h; and stage 7: drying at 20 to 25° C. for 1 to 2 h.
